(19) United States
(12) Patent Application Publication
(10) Pub. No.: US 2024/0262781 A1
(43) Pub. Date: Aug. 8, 2024

(54) DEPOLYMERIZABLE POLYOLEFINS FROM SOLUTION-PHASE POLYMERIZATION

(71) Applicants: Letian DOU, West Lafayette, IN (US); Qixuan Hu, West Lafayette, IN (US); Xuyi Luo, Lafayette, IN (US)

(72) Inventors: Letian DOU, West Lafayette, IN (US); Qixuan Hu, West Lafayette, IN (US); Xuyi Luo, Lafayette, IN (US)

(21) Appl. No.: 18/403,541

(22) Filed: Jan. 3, 2024

Related U.S. Application Data
(60) Provisional application No. 63/436,972, filed on Jan. 4, 2023.

(51) Int. Cl. C07C 67/475; C07C 67/58; C08F 22/10

(52) U.S. Cl. CPC C07C 67/475; C07C 67/58; C08F 22/10

(57) ABSTRACT

The disclosure relates to a process for obtaining a monomer or monomers from a non-crystalline random co-polymer, the process comprising the steps of heating the polymer in a solvent at a temperature and for a time sufficient to form the monomer or monomers. The disclosure also relates to the non-crystalline random copolymers.

### CROSS-REFERENCE TO RELATED APPLICATIONS

This application claims the benefit of U.S. Provisional Appl. No. 63/436,972, filed Jan. 4, 2023, which is incorporated by reference as if fully set forth herein.

### BACKGROUND

Single-use plastics triggered the material revolution of the 20th century, but also causes serious environmental pollution and drives petroleum consumption. Closed-loop circular utilization of plastics is of manifold significance, yet energy-intensive and poorly selective scission of the ubiquitous carbon-carbon (C—C) bonds in commercial polymers pose tremendous challenges to envisioned recycling and upcycling scenarios. Currently, there are few low-cost and scalable substitutes for typical polyolefin materials that are amenable to closed-loop circular utilization.

Recently, multiple catalytical depolymerization reactions of C—C bonded polymers via olefin metathesis were reported but with expensive Ru and Ir based transition metal catalysts. Meanwhile, a classic example poly(α-methyl styrene), exhibiting a weak C—C bond, has a ceiling temperature below 100° C. and the monomer can be fully recovered via pyrolysis. Despite that poly(α-methyl styrene) is too unstable for real applications, pyrolysis is still a convenient and low-cost chemical recycling approach for polymers. For instance, Chen and coworkers recently reported a bridged-ring polyamide material that can be circularly recyclable with a pyrolysis temperature of 300° C. Thus, an ideal C—C bond for chemically recyclable polymers should be strong enough for processing and application, and at the same time weak enough for depolymerization via pyrolysis.

In response to these challenges, recently a topochemical polymerization approach for creating elongated C—C bonds (1.58˜1.61 Å) in polymers with decreased bond dissociation energies (50˜70 kcal/mol) was demonstrated (FIG. 1A). These polymers exhibit rapid depolymerization within a desirable temperature range, while remaining remarkably stable under harsh conditions similar to traditional polyolefin materials. However, although these topochemically prepared polymer crystals are processable, their structural and property diversity as well as industrial-scale manufacturability are limited. This is not only because solid state polymerization takes place on specific molecular arrangement on the bulk surface that results in reactivity/reaction efficiency difficulties at a large scale, but also due to the cost of post-processing of polymer crystals into useful forms. In contrast, the majority of the existing polymers are produced via solution/melt polymerization and molded through injection molding, compression molding etc., where polymer crystals cannot be easily applied. Design and modification of the new polymer structures for extensive applications are also restricted in the topochemical approach. Single-crystal to single-crystal transformation requires finely controlled atom-atom position to create chemical bonds in the lattice. It is challenging to predict crystal structures of a designed monomer beforehand. Any modifications, including side chain modifications to the structure can lead to loss of photo reactivity of monomers. All these difficulties set obstacles to real-world applications of polymer single crystals, which prompted a reinvestigation of the possibility of synthesizing polymers via industry-compatible approaches (e.g., solution-phase free-radical polymerization) and examination of their depolymerizability.

### SUMMARY

The disclosure relates to a process for obtaining a monomer of formula (M-I) and/or a monomer of formula (M-II)

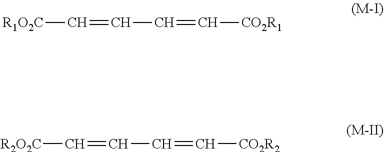

from a non-crystalline random co-polymer of formula (P-I)

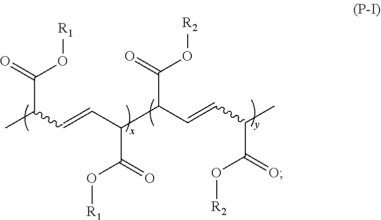

or
a monomer of formula (M-I):
wherein:
the wavy lines denote E-Z isomerism;
x indicates the mole percentage (mol %) of the repeating unit R₁O₂C—CH—CH═CH—CH—CO₂R₁ in P-I;
y indicates the mol % of the repeating unit R₂O₂C—CH—CH═CH—CH—CO₂R₂) in P-I, where x+y=100 with the proviso that if y=0, only the monomer (M-I) is obtained and if x=0, only the monomer (M-II) is obtained; and
R₁ and R₂ are not identical and are independently selected from the group consisting of alkyl and optionally substituted aryl-alkylene;
the process comprising the steps of heating the polymer of formula (P-I) in a solvent at a temperature and for a time sufficient to form M-I and/or M-II.

The disclosure also relates to a non-crystalline random co-polymer comprising a first repeating unit of the chemical formula (R₁O₂C—CH—CH═CH—CH—CO₂R₁)ₓ and second repeating unit of the chemical formula (R₂O₂C—CH—CH═CH—CH—CO₂R₂)_{y} wherein

x indicates the non-zero mole percentage (mol %) of the first repeating unit;
y indicates the non-zero mol % of the second repeating unit; and
R₁ and R₂ are not identical and are independently selected from the group consisting of alkyl and optionally substituted aryl-alkylene.

### DESCRIPTION OF THE DRAWINGS

The drawings illustrate generally, by way of example, but not by way of limitation, various embodiments discussed herein.

FIG. 1A is a scheme showing the design of recyclable free radical polymerized polyMEs inspired from recyclable topochemical polyME polymer crystal.

FIG. 1B is a scheme showing free radical polymerized polyME-Et conformational structures and their mole percentages, with the proposed thermal recycling product.

FIG. 1C is a quantitative ¹³C NMR spectrum of specific polyME-Et.

FIG. 1D is a plot showing the calculated bond dissociation energies (BDE) of different repeating unit connection C—C bond, with feasible depolymerization BDE represented by stars and unfeasible ones represented by triangle.

FIG. 2 is reactions schemes for depolymerization reactions of various amorphous polyME derivatives and co-polymers obtained from free-radical polymerization (left) and photographs of the actual polymers and recovered monomers.

FIG. 3 is differential scanning calorimetry (DSC) profiles of all PolyME derivatives (exothermic up). Arrows indicate the glass transition of the polymer.

FIG. 4 is a ¹H NMR spectrum of PolyME-Me (25° C., CDCl₃).

FIG. 5 is a ¹H NMR spectra of ME-B and ME-Me Copolymer (PolyME-Me3B7) with comparison of PolyME-Me and PolyME-B (25° C., CDCl₃).

FIG. 6A is photographs of the fresh ME-Me monomer, amorphous polymer, and recycled monomers showing the complete monomer-to-monomer cycle.

FIG. 6B is overlays of ¹H NMR spectra of fresh ME-Me (top) and recycled ME-Me (bottom) (25° C., CDCl₃).

FIG. 7A is photographs of the fresh ME-Et monomer, amorphous polymer, and recycled monomers showing the complete monomer-to-monomer cycle.

FIG. 7B is overlays of ¹H NMR spectra of fresh ME-Et (top) and recycled ME-Et (bottom) (25° C., CDCl₃).

FIG. 8A is photographs of the fresh ME-B monomer, amorphous polymer, and recycled monomers showing the complete monomer-to-monomer cycle.

FIG. 8B is overlays of ¹H NMR spectra of fresh ME-B (top) and recycled ME-B (bottom) (25° C., CDCl₃).

FIG. 9A scheme of ME monomers and polyMEs with corresponding polymerization and depolymerization conditions.

FIG. 9B is photographs showing polyME homopolymers and recovered monomers with the chemical structures of different R groups and abbreviations of the corresponding homopolymers. All scale bars are 5 mm.

FIG. 9C is a stress-strain curves for six polyME homopolymers: polyME-Me, polyME-2F, polyME-Cb, polyME-B, polyME-Et, and polyME-Pr.

FIG. 9D is stress-strain curves of polyME copolymers with the comparison of two pristine homopolymers.

FIGS. 10A-10B is a photograph of a laboratory large size synthesis of ME monomers.

FIG. 10C is a size exclusion chromatography (SEC) curves for the pristine polymer-Me7B3 and after injection molding samples.

FIG. 10D is a photograph of extruded polyME-Me3B7 filament from microcompounder and a 3D-printed Purdue “P” logo produced from polymer-Me7B3 filament.

FIG. 10E is a photograph of injection molding samples of polyME-Me7B3: tensile test bar (left); tensile test bar after mechanical testing (middle); a dinosaur toy made with silicone mold.

### DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

The availability of economic and scalable alternatives to common polyolefin materials with closed-loop circular usage remains scant, due to the energy-intensive and imprecise cleavage of the carbon-carbon (C—C) bonds that present substantial obstacles to potential recycling and upcycling strategies. The instant disclosure relates to, among other things, demonstration free-radical polymerization as a potent method for producing high molecular weight polymuconic ester (polyME) derivatives. Rapid depolymerization at a temperature of 250° C. can be achieved with various polymers, including six distinctive polymers and five copolymers, due to their intrinsically weakened C—C bonds. Modifying the side chains and copolymerization ratios allowed for mechanical property tuning of the polymers, achieving remarkable performance highlighting the potential for the polymers to serve as alternatives to commercial plastics. Furthermore, the smooth integration of conventional processing methods (injection molding, 3-D printing, etc.) and the bio-sourced precursor indicated the potential compatibility with large scale commercialization of these recyclable polymers. The instant disclosure demonstrates that free-radical polymerized polyMEs are promising for practical applications and large-scale industrialization with complete materials' circularity. Techno-economic analysis and supply chain-based life cycle assessment for the production of a representative polyME from biobased muconic acid estimate that the production could be economically and environmentally competitive with conventional acrylics. Furthermore, use of recycled material via depolymerization has the potential to further decrease the cost and impacts of polyME production.

The disclosure relates to a process for obtaining a monomer of formula (M-1) and/or a monomer of formula (M-II)

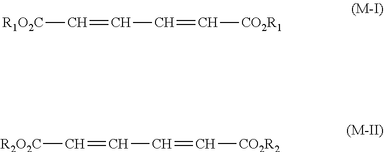

from a non-crystalline random co-polymer of formula (P-I)

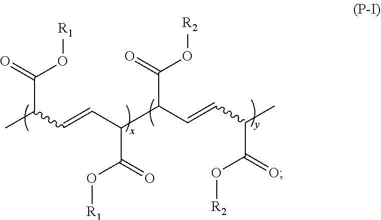

or
a monomer of formula (M-I):
wherein:
the wavy lines denote E-Z isomerism;
x indicates the mole percentage (mol %) of the repeating unit R₁O₂C—CH—CH═CH—CH—CO₂R₁ in P-I;
y indicates the mol % of the repeating unit R₂O₂C—CH—CH═CH—CH—CO₂R₂) in P-I, where x+y=100 with the proviso that if y=0, only the monomer (M-I) is obtained and if x=0, only the monomer (M-II) is obtained; and
R₁ and R₂ are not identical and are independently selected from the group consisting of alkyl and optionally substituted aryl-alkylene;
the process comprising the steps of heating the polymer of formula (P-I) in a solvent at a temperature and for a time sufficient to form M-I and/or M-II. The process can further comprise isolating M-I and/or M-II from solution by any suitable means. In some embodiments, the heating step can be carried out under an inert atmosphere.

Thus, for example, the process can comprise the steps of heating the polymer of formula (P-I) in a solvent under an inert atmosphere at a temperature of about 240° C. to about a temperature of about 270° C. for a time of about 30 minutes to about 60 minutes, cooling the solution to about room temperature; and isolating the M-I, M-II, or the mixture of M-I and M-II by chromatography.

The process can further comprise (re)polymerizing M-I and/or M-II to give P-I. The polymerization can be carried out using any suitable method including photochemically polymerize M-I and/or M-II above their melting temperature using ultraviolet (UV) light, or by radical polymerization (e.g., using a radical initiator, such as tert-butyl peroxide, for 48 hours of heating at 100° C., P-I can be obtained with yields of up to 70%).

The monomers of the formula (M-I) and (M-II), as well as the polymer of the formula (P-I) have double bonds that can have E- or Z-configurations. For example, monomer (M-I) can be the E,E isomer, substantially free of the E,Z and Z,Z isomers. Alternatively, the monomer (M-II) can be the E,E isomer, substantially free of the E,Z and Z,Z isomers. In some instances, monomer (M-I) can be the Z,Z isomer, substantially free of the E,Z and E, E isomers. Alternatively, the monomer (M-II) can be the Z,Z isomer, substantially free of the E,Z and E,E isomers. In still other instances, monomer (M-I) can be the E,Z isomer, substantially free of the Z,E; E,E; and Z,Z isomers. Alternatively, the monomer (M-II) can be the E,Z isomer, substantially free of the Z,E; E,E; and Z,Z isomers. In yet other instances, the monomer (M-I) can be the Z,E isomer, substantially free of the E,Z; E,E; and Z,Z isomers. Alternatively, the monomer (M-II) can be the Z, E isomer, substantially free of the E,Z; E,E; and Z,Z isomers.

The monomers of the formula (M-I) and (M-II) can be recovered from the polymer of the formula (P-I) at a monomer recover yield of at least about 50%, at least about 60%, at least about 70%, at least about 80%, from about 50% to about 90%, about 50% to about 100%, about 50% to about 75%, about 60% to about 80% or about 55% to about 85%.

In the processes described herein, R₁ and R₂ are each independently alkyl or R₁ and R₂ are each independently optionally substituted aryl-alkylene. For example, R₁ can be C₁-C₄ alkyl, C₁-C₆ alkyl, or C₁-C₈ alkyl, such as methyl, ethyl, n-propyl, isopropyl, n-butyl, isobutyl, pentyl, neopentyl, hexyl, heptyl, or octyl. In some specific examples, R₁ can be methyl, ethyl, n-propyl, n-butyl, or isobutyl; R₁ can be methyl or ethyl; or R₁ can be methyl. In some examples, R₁ is ethyl.

Alternatively, or in addition the aforementioned variations of R₁, R₂ can be C₁-C₆ alkyl, C₁-C₈ alkyl, or optionally substituted aryl-alkylene, with the proviso that R₁ and R₂ are different. Thus, for example, R₂ can be methyl, ethyl, n-propyl, n-butyl, isobutyl, pentyl, neopentyl, hexyl, heptyl, octyl, or optionally substituted aryl-alkylene. In some specific examples, R₂ can be methyl, ethyl, n-propyl, n-butyl, isobutyl, or optionally substituted aryl-alkylene. In other examples, R₂ can be optionally substituted aryl-alkylene, such as optionally substituted phenylalkyl, such as phenyl-C₁-C₄ alkylene. R₂ can therefore be phenyl-CH₂CH₂CH₂CH₂, phenyl-CH₂CH₂CH₂, phenyl-CH₂CH₂, or phenyl-CH₂, each of which is optionally substituted. In some examples, R₂ can be optionally substituted benzyl, such as benzyl.

The process described herein for obtaining M-I and/or M-II from P-I can be carried out in any suitable high boiling point solvent, such as any inert solvent with a boiling point of 240° C. or a boiling point of about 250° C. or higher. Non-limiting examples inert, high boiling point solvents include 1,3-dimethyltetrahydropyrimidin-2(1H)-one (DMPU) (246° C.), 2,5,8,11,14-pentaoxapentadecane (TEGDME; also known as tretraglyme) (275.3° C.), and diphenyl ether (258° C.). A preferred solvent to carry out the processes described herein is diphenyl ether.

The processes described herein for obtaining M-I and/or M-II from P-I can be carried out at any suitable temperature, such about 250° C. or higher, such as at about 240° C. to about 280° C. or about 255° C. to about 265° C. In some examples, the processes described herein can be carried out at about between 260° C. and 300° C.

The processes described herein for obtaining M-I and/or M-II from P-I can be carried out over any suitable amount of time, such as for at least about 15 minutes, at least about 20 minutes, at least about 30 minutes, at least about 45 minutes, or at least about 60 minutes. The processes described herein can be carried out, for example, for about 30 minutes, about 35 minutes, about 40 minutes, about 45 minutes, about 60 minutes, from about 15 minutes to about 60 minutes, about 30 minutes to about 60 minutes, about 30 minutes to about 45 minutes, or about 40 minutes to about 60 minutes.

The non-crystalline random co-polymer of formula (P-I):

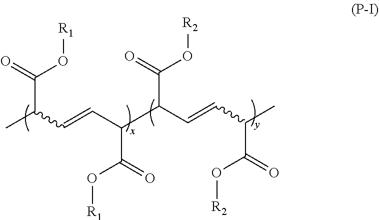

can have any suitable mol % values for x and y. For example, y can be 0 mol % to 100 mol %. In some instances, y is 0 mol %, such that the polymer (P-I) becomes a polymer having the repeating unit:

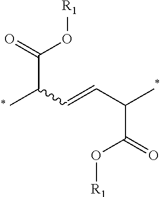

In some examples, y can be from about 5 mol % to 95 mol %, 10 mol % to about 90 mol %, 30 mol % to about 90 mol %, 30 mol % to about 70 mol %, about 20 mol % to about 80 mol %, about 40 mol % to about 80 mol %, about 40 mol % to about 75 mol %, about 50 mol % to about 80 mol %, about 50 mol % to about 75 mol %, 50 mol % to about 90 mol %, 60 mol % to about 80 mol %, or about 35 mol % to about 70 mol %. In some examples, y is about 70 mol %. In other examples, y can be 100 mol %.

In some examples, x can be from about 5 mol % to 95 mol %, 10 mol % to about 90 mol %, 30 mol % to about 90 mol %, 30 mol % to about 70 mol %, about 20 mol % to about 80 mol %, about 40 mol % to about 80 mol %, about 40 mol % to about 75 mol %, about 50 mol % to about 80 mol %, about 50 mol % to about 75 mol %, 50 mol % to about 90 mol %, 60 mol % to about 80 mol %, or about 35 mol % to about 70 mol %. In some examples, y is about 70 mol %. In other examples, y can be 100 mol %.

The disclosure also relates to a non-crystalline random co-polymer comprising a first repeating unit of the chemical formula (R₁O₂C—CH—CH═CH—CH—CO₂R₁)ₓ and second repeating unit of the chemical formula (R₂O₂C—CH—CH═CH—CH—CO₂R₂)_{y}

wherein
x indicates the non-zero mole percentage (mol %) of the first repeating unit;
y indicates the non-zero mol % of the second repeating unit; and
R₁ and R₂ are not identical and are independently selected from the group consisting of alkyl and optionally substituted aryl-alkylene.

In the non-crystalline random co-polymers described herein, R₁ and R₂ are each independently alkyl or R₁ and R₂ are each independently optionally substituted aryl-alkylene. For example, R₁ can be C₁-C₄ alkyl, C₁-C₆ alkyl, or C₁-C₈ alkyl, such as methyl, ethyl, n-propyl, isopropyl, n-butyl, isobutyl, pentyl, neopentyl, hexyl, heptyl, or octyl. In some specific examples, R₁ can be methyl, ethyl, n-propyl, n-butyl, or isobutyl; R₁ can be methyl or ethyl; or R₁ can be methyl. In some examples, R₁ is ethyl.

Alternatively, or in addition the aforementioned variations of R₁, R₂ can be C₁-C₆ alkyl, C₁-C₈ alkyl, or optionally substituted aryl-alkylene, with the proviso that R₁ and R₂ are different. Thus, for example, R₂ can be methyl, ethyl, n-propyl, n-butyl, isobutyl, pentyl, neopentyl, hexyl, heptyl, octyl, or optionally substituted aryl-alkylene. In some specific examples, R₂ can be methyl, ethyl, n-propyl, n-butyl, isobutyl, or optionally substituted aryl-alkylene. In other examples, R₂ can be optionally substituted aryl-alkylene, such as optionally substituted phenylalkyl, such as phenyl-C₁-C₄ alkylene. R₂ can therefore be phenyl-CH₂CH₂CH₂CH₂, phenyl-CH₂CH₂CH₂, phenyl-CH₂CH₂, or phenyl-CH₂, each of which is optionally substituted. In some examples, R₂ can be optionally substituted benzyl, such as benzyl.

In some examples of the non-crystalline random co-polymers described herein. In some examples, y can be from about 5 mol % to 95 mol %, 10 mol % to about 90 mol %, 30 mol % to about 90 mol %, 30 mol % to about 70 mol %, about 20 mol % to about 80 mol %, about 40 mol % to about 80 mol %, about 40 mol % to about 75 mol %, about 50 mol % to about 80 mol %, about 50 mol % to about 75 mol %, 50 mol % to about 90 mol %, 60 mol % to about 80 mol %, or about 35 mol % to about 70 mol %. In some examples, y is about 70 mol %.

In some examples, x can be from about 5 mol % to 95 mol %, 10 mol % to about 90 mol %, 30 mol % to about 90 mol %, 30 mol % to about 70 mol %, about 20 mol % to about 80 mol %, about 40 mol % to about 80 mol %, about 40 mol % to about 75 mol %, about 50 mol % to about 80 mol %, about 50 mol % to about 75 mol %, 50 mol % to about 90 mol %, 60 mol % to about 80 mol %, or about 35 mol % to about 70 mol %. In some examples, y is about 70 mol %.

The non-crystalline random co-polymers described herein, such as those made by the processes described herein, can be thermoplastic. In other instances, non-crystalline random co-polymers described herein, such as those made by the processes described herein, can be elastomers, especially when y is 0 and each R₁ is, for example, a C₃-alkyl group, such as n-propyl.

The non-crystalline random co-polymers described herein, such as those made by the processes described herein, can have any suitable physical properties including a Young's Modulus, a glass transition temperature, polydispersity index, weight average molecular weight (Mw), number average molecular weight (MN); ultimate strength, and strain at break. For example, the non-crystalline random co-polymers described herein, such as those made by the processes described herein, can have a Young's Modulus of from about 0.5 GPa to about 1.6 GPa, about 0.5 GPa to about 1 GPa, about 0.75 GPa to about 1.5 GPa, about 0.9 GPa to about 1.6 GPa or about 0.8 GPa to about 1.5 GPa. Alternatively, or in addition, the non-crystalline random co-polymers described herein, such as those made by the processes described herein, can have a glass transition temperature (T_{g}) of from about −17° C. to about 60° C., 0° C. to about 30° C., about 5° C. to about 55° C. or about 10° C. to about 60° C. Alternatively, or in addition, the non-crystalline random co-polymers described herein, such as those made by the processes described herein, can have a polydispersity index (PDI) of from about 1.5 to about 1.9. Alternatively, or in addition, the non-crystalline random co-polymers described herein, such as those made by the processes described herein, can have a weight average molecular weight (Mw) of from about 100 kDa to about 600 kDa. Alternatively, or in addition, the non-crystalline random co-polymers described herein, such as those made by the processes described herein, can have a number average molecular weight (MN) of from about 50 kDa to about 400 kDa. Alternatively or in addition, the non-crystalline random co-polymers described herein, such as those made by the processes described herein, can have an ultimate strength of about 1 MPa to about 45 MPa, about 1 MPa to about 10 MPa, about 5 MPa to about 25 MPa, about 10 MPa to about 30 MPa, about 30 MPa to about 42 MPa, about 2 MPa to about 4 MPa, about 3 MPa to about 6 MPa, or about 15 MPa to about 25 MPa. Alternatively or in addition, the non-crystalline random co-polymers described herein, such as those made by the processes described herein, can have a strain at break from about 2% to about 2500%, about 10% to about 100%, about 50% to about 200%, about 150% to about 400%, about 250% to about 800%, about 300% to about 1500%, about 500% to about 2500%, about 2% to about 10%, about 2% to about 5%, about 1000% to about 2500%, about 1500% to about 2200%, about 200% to about 600%, about 300% to about 500%, about 450% to about 550%, about 10% to about 25%, about 10% to about 20% or about 15% to about 25%.

Since the non-crystalline random co-polymers described herein, such as those made by the processes described herein, have double bonds in their skeleton, they can be crosslinked, if so desired, by using any suitable method including using elemental sulfur in a process analogous to vulcanization of rubber with sulfur, thiol-ene click crosslinking, and transesterification of side chain using diols (e.g., ethylene glycol) or triols (e.g., glycerol). The crosslinking can be irreversible or reversible such that one can still access monomers (M-I) and (M-II) from crosslinked (P-I) by employing the methods described herein. Thus, for example, crosslinked (P-I) can be subjected to conditions that reverse the crosslinking to arrive at (P-I) (e.g., reduction of —S—S— bonds when the crosslinking includes —S—S— bonds). Then, (P-I) can be obtaining a monomer of formula (M-I) and/or a monomer of formula (M-II) by the methods described herein.

While the concepts of the present disclosure are illustrated and described in detail in the description herein, results in the description are to be considered as exemplary and not restrictive in character; it being understood that only illustrative embodiments are shown and described and that all changes and modifications that come within the spirit of the disclosure are desired to be protected.

As used herein, the following terms and phrases have the meanings set forth below. Unless defined otherwise, all technical and scientific terms used herein have the same meaning as commonly understood to one of ordinary skill in the art.

In the present disclosure the term “about” can allow for a degree of variability in a value or range, for example, within 10%, within 5%, or within 1% of a stated value or of a stated limit of a range. When the term “about” is used for both the upper and lower limits of a range, such as “about X to about Y”, its use is to be understood to also include “X to about Y” and “about X to Y.” In the present disclosure the term “substantially” can allow for a degree of variability in a value or range, for example, within 90%, within 95%, 99%, 99.5%, 99.9%, 99.99%, or at least about 99.999% or more of a stated value or of a stated limit of a range. In the present disclosure, “substantially free of X” indicates that a compound or material substantially free of X has less than about 10%, less than about 9%, less than about 8%, less than about 7%, less than about 6%, less than about 5%, less than about 4%, less than about 3%, less than about 2%, less than about 1%, less than about 0.5%, or less than about 0.1% of X in the material. The percentage may be a weight % or a mol % and will be understood based on the context of its use.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

As used herein, the term “alkyl” includes a chain of carbon atoms, which is optionally branched. It is to be further understood that in certain embodiments, alkyl is advantageously of limited length, including C₁-C₂₄, C₁-C₁₂, C₁-C₃, C₁-C₆, and C₁-C₄, and C₂-C₂₄, C₂-C₁₂, C₂-C₈, C₂-C₆, and C₂-C₄, and the like Illustratively, such particularly limited length alkyl groups, including C₁-C₈, C₁-C₆, and C₁-C₄, and C₂-C₈, C₂-C₆, and C₂-C₄, and the like may be referred to as lower alkyl. In embodiments described herein, it is to be understood, in each case, that the recitation of alkyl refers to alkyl as defined herein, and optionally lower alkyl. Illustrative alkyl, groups are, but not limited to, methyl, ethyl, n-propyl, isopropyl, n-butyl, isobutyl, sec-butyl, tert-butyl, pentyl, 2-pentyl, 3-pentyl, neopentyl, hexyl, 2-hexyl, 3-hexyl, heptyl, 2-heptyl, octyl, 2-octyl, and the like.

As used herein, the term “alkylene” includes a divalent chain of carbon atoms, which is optionally branched. It is to be further understood that in certain embodiments, alkylene is advantageously of limited length, including C₁-C₂₄, C₁-C₁₂, C₁-C₈, C₁-C₆, and C₁-C₄, and C₂-C₂₄, C₂-C₁₂, C₂-C₈, C₂-C₆, and C₂-C₄, and the like. Illustratively, such particularly limited length alkylene groups, including C₁-C₈, C₁-C₆, and C₁-C₄, and C₂-C₈, C₂-C₆, and C₂-C₄, and the like may be referred to as lower alkylene. In embodiments described herein, it is to be understood, in each case, that the recitation of alkylene, refers to alkylene, as defined herein, and optionally lower alkylene. Illustrative alkylene groups are, but not limited to, methylene, ethylene, n-propylene, isopropylene, n-butylene, isobutylene, sec-butylene, pentylene, 1,2-pentylene, 1,3-pentylene, hexylene, heptylene, octylene, and the like.

As used herein, the term “aryl” includes monocyclic and polycyclic aromatic carbocyclic groups, each of which may be optionally substituted. Illustrative aromatic carbocyclic groups described herein include, but are not limited to, phenyl, naphthyl, and the like.

As used herein the term aryl-alkylene indicates an alkylene group in which one of its two valences is attached to the aryl group.

The term “optionally substituted” as used herein includes the replacement of hydrogen atoms with other substituents on the radical that is optionally substituted. Illustrative substituents include, but are not limited to, halogen, hydroxy, alkyl, including C₁-C₆ alkyl, alkoxy, including C₁-C₆ alkoxy, haloalkyl, including C₁-C₆ haloalkyl and CF₃, cyano, nitro, —CO₂R⁴, or —CONR⁵R⁶, where R⁴, R⁵, and R⁶ are each independently selected in each occurrence from the group consisting of C₁-C₆ alkyl, and aryl-C₁-C₆ alkylene. It is to be understood that the term “optionally substituted” includes the option where no substituent is present.

The following clauses disclose several non-limiting embodiments of the disclosure.

Clause 1. A process for obtaining a monomer of formula (M-I) and/or a monomer of formula (M-II)

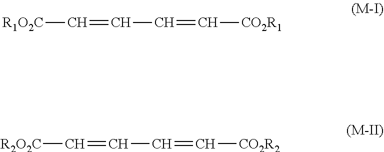

from a non-crystalline random co-polymer of formula (P-I)

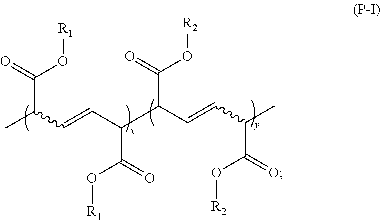

or
a monomer of formula (M-I):
wherein:
x indicates the mole percentage (mol %) of the repeating unit R₁O₂C—CH—CH═CH—CH—CO₂R₁ in P-I;
y indicates the mol % of the repeating unit R₂O₂C—CH—CH═CH—CH—CO₂R₂) in P-I, where x+y=100 with the proviso that if y=0, only the monomer (M-I) is obtained and if x=0, only the monomer (M-II) is obtained; and
R₁ and R₂ are not identical and are independently selected from the group consisting of alkyl and optionally substituted aryl-alkylene;
the process comprising the steps of heating the polymer of formula (P-I) in a solvent under an inert atmosphere at a temperature of about 240° C. to about a temperature of about 270° C. for a time of about 30 minutes to about 60 minutes, cooling the solution to about room temperature; and isolating the M-I, M-II, or the mixture of M-I and M-II by chromatography.

Clause 2. The process of clause 1, wherein R₁ and R₂ are each independently alkyl or R₁ and R₂ are each independently optionally substituted aryl-alkylene.

Clause 3. The process of clause 1 or 2, wherein R₁ is C₁-C₄ alkyl, C₁-C₆ alkyl, or C₁-C₈ alkyl.

Clause 4. The process of any preceding clause, wherein R₁ is methyl, ethyl, n-propyl, isopropyl, n-butyl, isobutyl, pentyl, neopentyl, hexyl, heptyl, or octyl.

Clause 5. The process of any preceding clause, wherein R₁ is methyl, ethyl, n-propyl, n-butyl, or isobutyl.

Clause 6. The process of any preceding clause, wherein R₁ is methyl or ethyl.

Clause 7. The process of any preceding clause, wherein R₁ is methyl.

Clause 8. The process of any of clauses 1-6, wherein R₁ is ethyl.

Clause 9. The process of any preceding clause, wherein R₂ is C₁-C₆ alkyl, C₁-C₈ alkyl, or optionally substituted aryl-alkylene, with the proviso that R₁ and R₂ are different.

Clause 10. The process of any preceding clause, wherein R₂ is methyl, ethyl, n-propyl, n-butyl, isobutyl, pentyl, neopentyl, hexyl, heptyl, octyl, or optionally substituted aryl-alkylene.

Clause 11. The process of any preceding clause, wherein R₂ is methyl, ethyl, n-propyl, n-butyl, isobutyl, or optionally substituted aryl-alkylene.

Clause 12. The process of any preceding clause, wherein R₂ is optionally substituted aryl-alkylene.

Clause 13. The process of any preceding clause, wherein R₂ is optionally substituted phenylalkyl.

Clause 14. The process of any preceding clause, wherein R₂ is optionally substituted phenyl-C₁-C₄ alkylene.

Clause 15. The process of any preceding clause, wherein R₂ is phenyl-CH₂CH₂CH₂CH₂, phenyl-CH₂CH₂CH₂, phenyl-CH₂CH₂, or phenyl-CH₂, each of which is optionally substituted.

Clause 16. The process of any preceding clause, wherein R₂ is optionally substituted benzyl.

Clause 17. The process of any preceding clause, wherein R₂ is benzyl.

Clause 18. The process of any preceding clause, wherein the solvent selected from group consisting of 1,3-dimethyltetrahydropyrimidin-2(1H)-one (DMPU), 2,5,8,11,14-Pentaoxapentadecane (TEGDME), and diphenyl ether.

Clause 19. The process of any preceding clause, wherein the solvent is diphenyl ether.

Clause 20. The process of any preceding clause, wherein the temperature is about 240° C. to about 280° C.

Clause 21. The process of any preceding clause, wherein the temperature is about 260° C.

Clause 22. The process of any preceding clause, wherein the time is about 45 minutes.

Clause 23. The process of any preceding clause, wherein y is 0 mol % to 100 mol %.

Clause 24. The process of clause 23, wherein y is 0 mol %.

Clause 25. The process of clause 23, wherein y is about 70 mol %.

Clause 26. The process of clause 23, wherein y is 100 mol %.

Clause 27. The process of any preceding clause, wherein the obtained monomer (M-I) is the E,E isomer, substantially free of the E,Z and Z,Z isomers.

Clause 28. The process of any preceding clause, wherein the obtained monomer (M-II) is the E,E isomer, substantially free of the E,Z and Z,Z isomers.

Clause 29. A non-crystalline random co-polymer comprising a first repeating unit of the chemical formula (R₁O₂C—CH—CH═CH—CH—CO₂R₁)ₓ and second repeating unit of the chemical formula (R₂O₂C—CH—CH═CH—CH—CO₂R₂)_{y} wherein
x indicates the non-zero mole percentage (mol %) of the first repeating unit;
y indicates the non-zero mol % of the second repeating unit; and
R₁ and R₂ are not identical and are independently selected from the group consisting of alkyl and optionally substituted aryl-alkylene.

Clause 30. The non-crystalline random co-polymer of clause 29, wherein R₁ and R₂ are independently alkyl or R₁ and R₂ are independently optionally substituted aryl-alkylene.

Clause 31. The random co-polymer of clause 29 or 30, wherein R₁ is C₁-C₄ alkyl, C₁-C₆ alkyl, or C₁-C₈ alkyl.

Clause 32. The non-crystalline random co-polymer of any of clauses 29-31, wherein R₁ is methyl, ethyl, n-propyl, n-butyl, isobutyl, n-pentyl, neopentyl, n-hexyl, n-heptyl, or n-octyl.

Clause 33. The non-crystalline random co-polymer of any of clauses 29-32, wherein R₁ is methyl, ethyl, n-propyl, n-butyl, or isobutyl.

Clause 34. The non-crystalline random co-polymer of any of clauses 29-33, wherein R₁ is methyl or ethyl.

Clause 35. The non-crystalline random co-polymer of any of clauses 29-34, wherein R₁ is methyl.

Clause 36. The non-crystalline random co-polymer of any of clauses 29-34, wherein R₁ is ethyl.

Clause 37. The non-crystalline random co-polymer of any of clauses 29-36, wherein R₂ is C₁-C₆ alkyl, C₁-C₈ alkyl, or optionally substituted aryl-alkylene.

Clause 38. The non-crystalline random co-polymer of any of clauses 29-37, wherein R₂ is methyl, ethyl, n-propyl, n-butyl, isobutyl, n-pentyl, neopentyl, n-hexyl, n-heptyl, n-octyl, or optionally substituted aryl-alkylene.

Clause 39. The non-crystalline random co-polymer of any of clauses 29-38, wherein R₂ is methyl, ethyl, n-propyl, n-butyl, isobutyl, or optionally substituted aryl-alkylene.

Clause 40. The non-crystalline random co-polymer of any of clauses 29-39, wherein R₂ is optionally substituted aryl-alkylene.

Clause 41. The non-crystalline random co-polymer of any of clauses 29-40, wherein R₂ is optionally substituted phenyl-alkylene.

Clause 42. The non-crystalline random co-polymer of any of clauses 29-41, wherein R₂ is optionally substituted phenyl-C₁-C₄ alkylene.

Clause 43. The non-crystalline random co-polymer of any of clauses 29-42, wherein R₂ is phenyl-CH₂CH₂CH₂CH₂, phenyl-CH₂CH₂CH₂, phenyl-CH₂CH₂, or phenyl-CH₂, each of which is optionally substituted.

Clause 44. The non-crystalline random co-polymer of any of clauses 29-43, wherein R₂ is optionally substituted benzyl.

Clause 45. The non-crystalline random co-polymer of any of clauses 29-44, wherein R₂ is benzyl.

Clause 46. The non-crystalline random co-polymer of any of clauses 29-45, wherein y is 5 mol % to 95 mol %.

Clause 47. The non-crystalline random co-polymer of any of clauses 29-46, wherein y is about 10 mol % to about 90 mol %.

Clause 48. The non-crystalline random co-polymer of any of clauses 29-47, wherein y is about 20 mol % to about 80 mol %.

Clause 49. The non-crystalline random co-polymer of any of clauses 29-48, wherein y is about 30 mol % to about 70 mol %.

Clause 50. The non-crystalline random co-polymer of any of clauses 29-49, wherein y is about 50 mol % to about 90 mol %.

Clause 51. The non-crystalline random co-polymer of any of clauses 29-50, wherein y is about 60 mol % to about 80 mol %.

Clause 52. The non-crystalline random co-polymer of any of clauses 29-51, wherein y is about 70 mol %.

Clause 53. The non-crystalline random co-polymer of any of clauses 29-52, wherein the non-crystalline random co-polymer is a thermoplastic.

Clause 54. The non-crystalline random co-polymer of any of clauses 29-53, wherein the non-crystalline random co-polymer has a Young's Modulus of about 0.5 to about 1.6.

Clause 55. The non-crystalline random co-polymer of any of clauses 29-54, wherein the non-crystalline random co-polymer has a weight average molecular weight (Mw) of from about 100 kDa to about 600 kDa.

Clause 56. The non-crystalline random co-polymer of any of clauses 29-55, wherein the non-crystalline random co-polymer has a number average molecular weight (MN) of from about 50 kDa to about 400 kDa.

Clause 57. The non-crystalline random co-polymer of any of clauses 29-56, wherein the non-crystalline random co-polymer has an ultimate strength of from about 1 MPa to about 45 MPa.

Clause 58. The non-crystalline random co-polymer of any of clauses 29-57, wherein the non-crystalline random co-polymer has a strain at break of from about 2% to about 2500%.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “substantially no” as used herein refers to less than about 30%, 25%, 20%, 15%, 10%, 5%, 3%, 2%, 1%, 0.5%, 0.1%, 0.05%, 0.001%, or at less than about 0.0005% or less or about 0% or 0%.

Those skilled in the art will appreciate that many modifications to the embodiments described herein are possible without departing from the spirit and scope of the present disclosure. Thus, the description is not intended and should not be construed to be limited to the examples given but should be granted the full breadth of protection afforded by the appended claims and equivalents thereto. In addition, it is possible to use some of the features of the present disclosure without the corresponding use of other features. Accordingly, the foregoing description of or illustrative embodiments is provided for the purpose of illustrating the principles of the present disclosure and not in limitation thereof and can include modification thereto and permutations thereof.

### EXAMPLES

The disclosure can be better understood by reference to the following examples which are offered by way of illustration. The disclosure is not limited to the examples given herein.

### Example 1

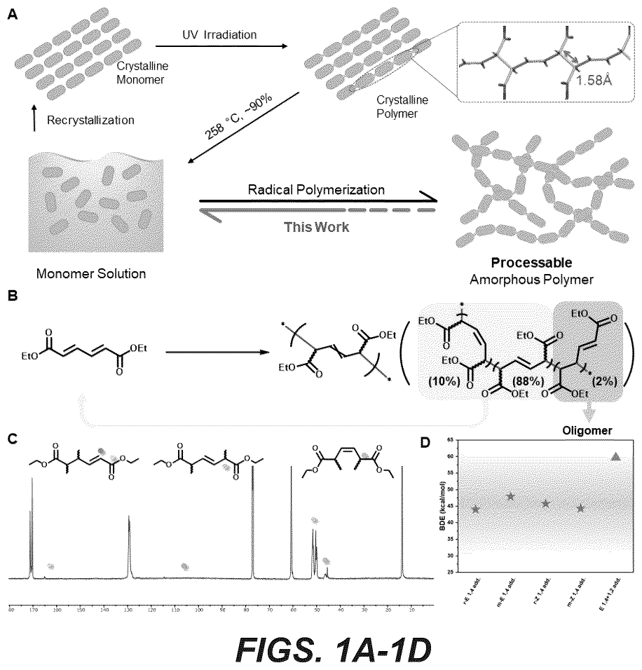

Two important fundamental questions arise naturally. First, can high-molecular weight polymers and co-polymers be synthesized via conventional approaches (instead of via topochemical polymerization)? Second, will the resulting polymers still maintain the elongated and weakened C—C bond similar to the topochemically-prepared counterparts (FIG. 1B)? To find answers to these questions, an integrated experimental and computational study on a series of polymuconate ester (polyME) derivatives was undertaken.

The computational results for the polyME structure surprisingly suggest a relatively low bond dissociation energy for the C—C bond between monomers even without the restriction in crystal lattices, making traditionally synthesized amorphous polyME an intriguing candidate for circular utilization (6). Initial solid-state depolymerization experiments show that a polyME derivative, polyME-Et, synthesized from free-radical polymerization, indeed started to depolymerize at around 230° C. This inspired further investigation of the polymerization and depolymerization dynamics of other polyME derivatives with different side chain chemistries and functionalities.

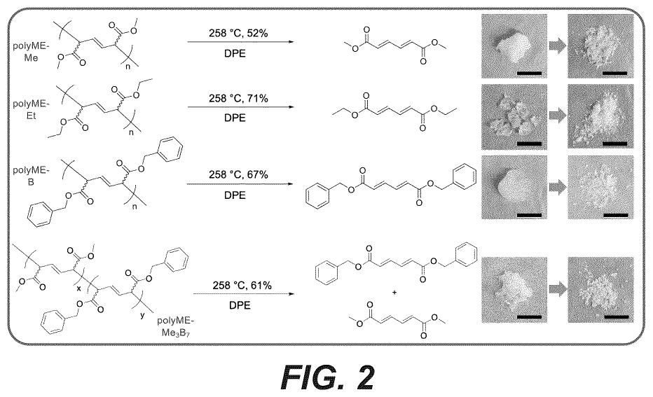

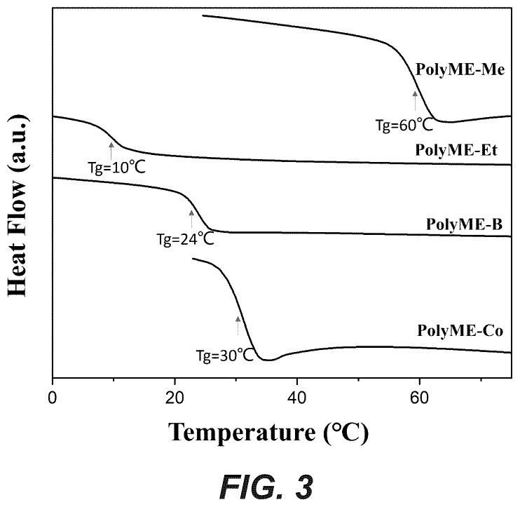

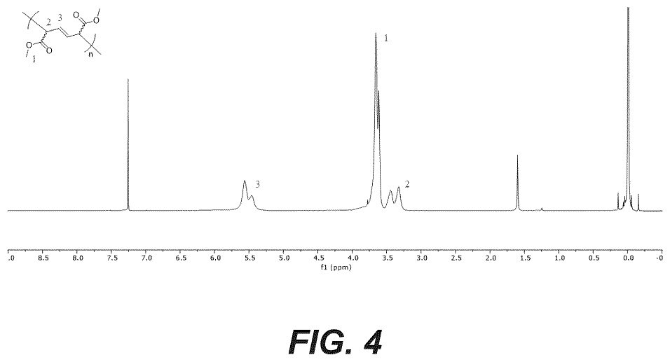

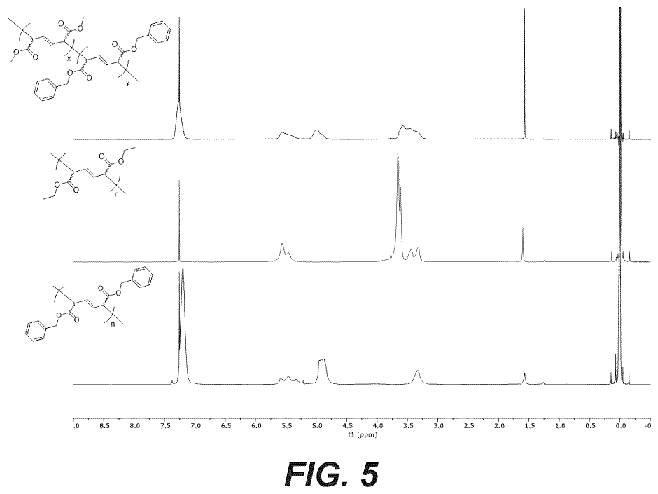

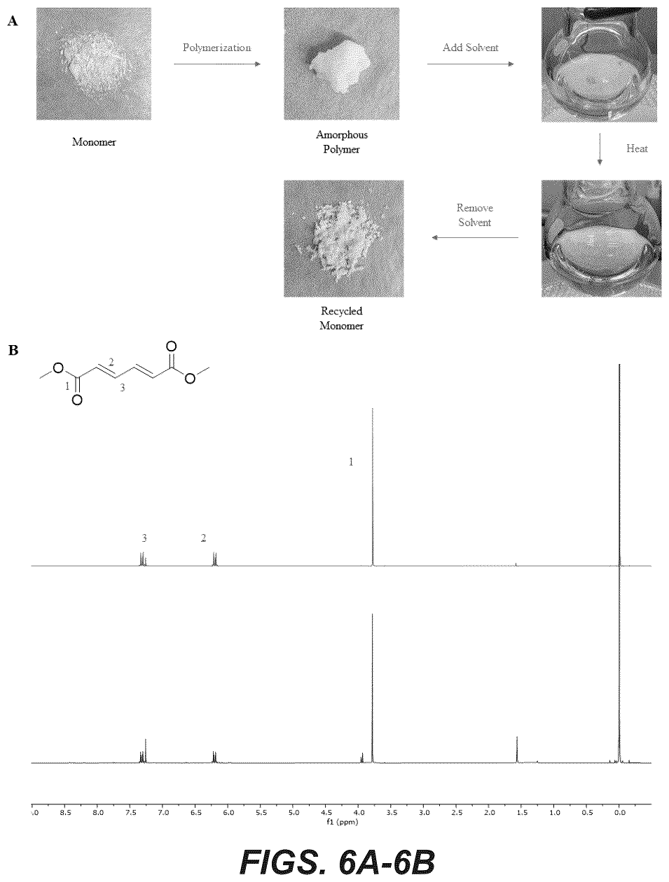

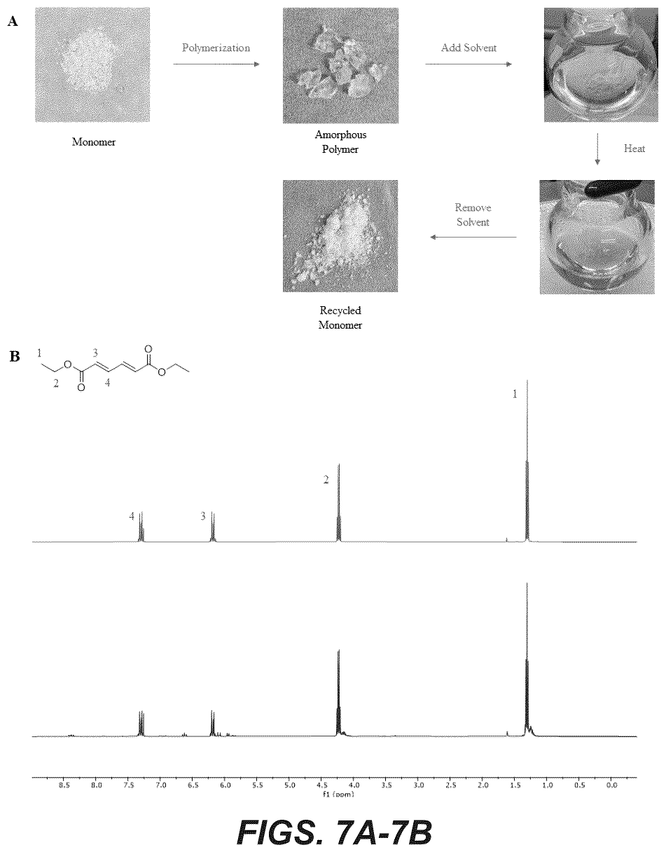

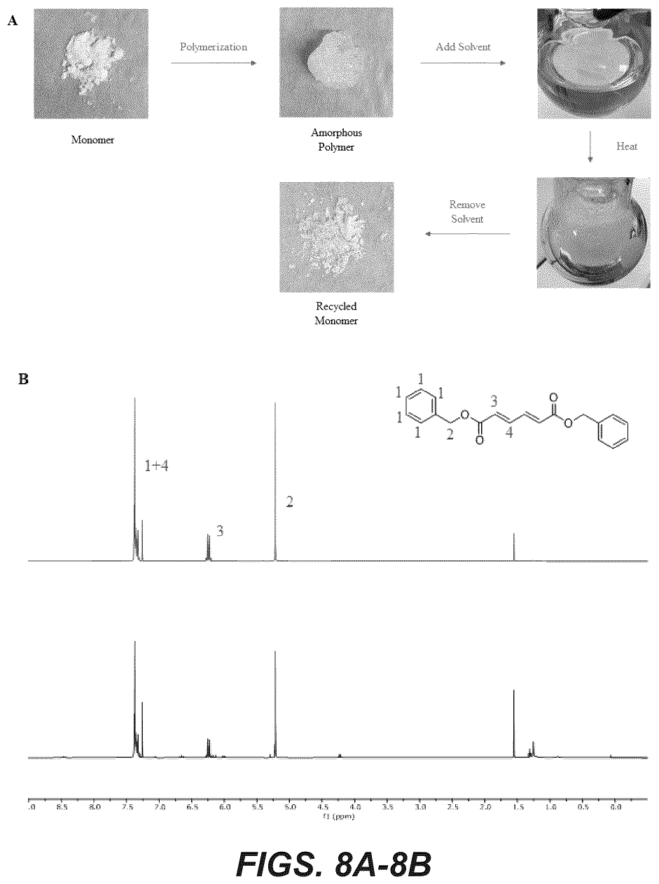

Three polyME derivatives featuring a methyl, an ethyl, and a benzyl sidechain (-Me, -Et and —B, FIG. 2) were prepared via a modified free-radical polymerization method for experimental validation of polymerization and depolymerization. Preliminary work on homopolymers was carried out by Matsumoto et al. but a comprehensive understanding has not been achieved and copolymerization has not been realized. Importantly, ME-Me and ME-B monomers do not undergo topochemical polymerization in the single crystals so that the corresponding polymer crystals are not yet accessible. Free-radical polymerization offers greater structural and property tunability. Furthermore, random copolymers composed of 70 mol % ME-B and 30 mol % ME-Me repeating units (polyME-Me3B7) was prepared. Copolymers are commonly produced for tuning polymer properties. Using a small amount of a radical initiator, tert-butyl peroxide, and 48 hours of heating, these polymers were obtained with yields of up to 70%. The number average molecular weights (MN) of these polymers are between 110 kDa to 306 kDa, while their polydispersity is between 1.55-1.72 (Table 1). The molecular weights of these polyME polymers are comparable to those of commercial PMMAs and thus can potentially serve as thermoplastics. The ratio of repeating units in polyME-Me3B7 was confirmed by ¹H-NMR.

TABLE 1
Representative weight average molecular weights (M_{W}),
number average molecular weights (M_{N}), polydispersity
index, and the glass transition temperature of polyMEs.

| Polymers | M_{w} (kDa) | Mₙ (kDa) | PDI | T_{g} (° C.) |
|---|---|---|---|---|
| PolyME-Me | 299 | 184 | 1.63 | 60 |
| PolyME-B | 521 | 306 | 1.70 | 24 |
| PolyME-Et | 188 | 110 | 1.72 | 10 |
| PolyME-Me3B7 | 272 | 176 | 1.55 | 31 |

Differential scanning calorimetry (DSC) was used to determine possible phase transitions of polyMEs during heating-cooling cycles. No melting could be found within a range of −40 to 180° C., while glass transition temperatures (T_{g}) of PolyMEs were detected between 10-60° C. The rigid polyME-Me has a highest T_{g} of 60° C. Surprisingly, polyME-Et with only one more carbon in the side chains exhibits a rather low T_{g} of 10° C. PolyME-B has a moderate T_{g} of 24° C. close to the room temperature. The trend of polyMEs' glass transition temperatures based on different side chains are analogous with poly(methyl acrylate) derivatives (9). The glass transition temperature of polyME-Me3B7 is 31° C., which agrees well with the Fox Equation (predicted T_{g} 33° C.). These results indicate that free-radical polymerization is an effective approach to synthesize a wide range of high quality polyME derivatives including copolymers.

Depolymerization experiments were conducted through a solvent-based depolymerization approach established previously. All tested polymers were converted to monomers at a temperature of 258° C. within 20 minutes in diphenyl ether (DPE) solution at reflux. Monomers were initially recovered in cis-trans configuration isomer forms. These mixtures of isomers can be isolated or transformed to a thermodynamically more stable (trans-, trans-) isomer through a simple iodine treatment. Off-white monomer powders were recovered with over 70% yield for depolymerization of polyME-B and polyME-Et and with 52% yield for depolymerization of polyME-Me. Compared to the reported depolymerization from polymer crystals, free-radical polymerization methods appear to lead to slightly more side reactions during depolymerization. The predominant side products are Diels-Alder addition of monomers. Polymer structural defects generated from radical polymerizations may also contribute to side products. Among the tested polymers, polyME-Me has a lowest depolymerization recovery of 52%. This may be due to relatively unstable nature of the ME-Me monomer. A polyME-Me3B7 copolymer consisting of ME-Me and ME-B alleviated this problem and brought the overall monomer recovery yield up to 61%. The depolymerization results support the theoretical studies that were performed indicating that the C—C bonds between monomer subunits in the polyME derivatives are intrinsically weak, independent of the way they were polymerized and their crystalline/amorphous state.

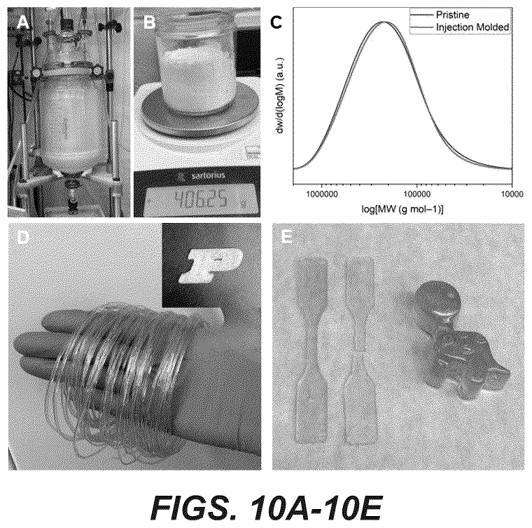

The fundamental mechanical properties of polyMEs were characterized by tensile stress-strain tests. Bulk polyME derivatives was extruded from a microcompounder and subsequently heat-press molded by a vacuum heat-pressing machine to obtain the standard dog-bone shaped specimens for testing (FIG. 10). The polymer specimens are optically transparent due to their amorphous nature. All tensile stress-strain test results are summarized in Table 2.

TABLE 2
Statistics of the tensile stress-strain tests of polymers.

| | Young's | Ultimate | Strain at |
|---|---|---|---|
| | Modulus | strength | break |
| Polymers | (Gpa) | (Mpa) | (%) |
| PolyME-Me | 1.52 ± 0.04 | 38 ± 2.1 | 3.5 ± 0.4 |
| #1 | 1.59 | 35.4 | 3.6 |
| #2 | 1.52 | 41.0 | 3.9 |
| #3 | 1.54 | 39.2 | 3.8 |
| #4 | 1.46 | 37.0 | 2.8 |
| PolyME-Et | 0.009 ± 0.003 | 2.3 ± 0.3 | 2170 ± 140 |
| #1 | 0.006 | 2.3 | 2140 |
| #2 | 0.008 | 2.0 | 2200 |
| #3 | 0.013 | 2.7 | 1870 |
| PolyME-B | 0.50 ± 0.02 | 4.0 ± 0.4 | 462 ± 21 |
| #1 | 0.48 | 4.1 | 447 |
| #2 | 0.52 | 4.6 | 450 |
| #3 | 0.51 | 3.9 | 454 |
| #4 | 0.48 | 3.5 | 498 |
| PolyME- | 0.80 ± 0.06 | 20.7 ± 1.7 | 19.4 ± 1.1 |
| Me3B7 | | | |
| #1 | 0.72 | 21.8 | 17.8 |
| #2 | 0.85 | 18.3 | 20.1 |
| #3 | 0.83 | 22.0 | 20.3 |

The stress σ and strain & were calculated using equations shown equation (1):

$$σ = \frac{F}{{A}_{0}} = \frac{F}{H \times W} , ε = \frac{L - {L}_{0}}{{L}_{0}} \qquad ( 1 )$$

The axial length L₀, width W, and thickness H were measured by a digital caliper.

Young's modulus was calculated by slope of the linear interval of the stress-strain curve, i.e., o/E. Ultimate strength and Strain at break are the stress and strain at the point of failure, respectively.

PolyME-Me exhibits a large Young's modulus of 1.59 Gpa (avg. 1.52 Gpa) and a ultimate strength of 39 Mpa (avg. 38 Mpa). On the other hand, PolyME-B exhibits a large elongation with strain of break at 498% (avg. 462%), while showing an average Young's modulus of 0.49 Gpa. polyME-Et exhibits an even larger elongation with strain of break of 1870% (avg. 2170%), while showing a low average Young's modulus of 0.009 GPa, which is consistent with its low T_{g}. This result suggests its potential application as an elastomer after crosslinking, as the polymer backbone is indeed the same as extensively studied butadiene-based natural or synthetic rubber (11). The distinct mechanical properties derived from different polyME derivatives demonstrate the broad application prospects of these recyclable polymers available by modifying the side chain chemistry. Modification of the resulting polymer mechanical properties via copolymerization is also possible. For example, the copolymer polyME-Me3B7 inherits the characteristics from both polyME-Me and polyME-B, with a good Young's modulus of 0.80 Gpa and a moderate elongation of 19.4%. This result suggests that mechanical properties of these copolymers may not vary linearly with the monomer ratios because it appears that the 30% polyME-Me portion dominates the mechanical properties of polyME-Me3B7. To demonstrate polyME's thermoplasticity in potential industrial applications, a hundred-gram scale synthesis of polyME-B was performed for further processing. The polymer filaments were subsequently extruded under a moderate temperature of 100° C. with a microcompounder with a diameter of ˜1.7 mm. The filament was finally fed into a 3D printer and a Purdue “P” logo was printed.

The results shown here indicate that free-radical polymerization is an effective way to produce high-molecular-weight polymuconate derivatives and copolymers. Similarly, to topochemically prepared polymer crystals, the polymers described here are depolymerizable under thermolysis conditions at about 250° C. in a high-boiling point solvent such as diphenyl ether. Perhaps due to their intrinsically elongated and weakened C—C bonds. Although only free-radical polymerization was examined here, it is believed that other approaches (such as cationic, anionic, or coordination polymerization) should also lead to similar polymers with a weakened C—C bond. Surprisingly, by changing the side groups, the mechanical properties of these polymers and/or copolymers can be tuned from hard and brittle to soft and stretchable. The chemistry described here is scalable and the processing of these materials is fully compatible with existing industry infrastructures, making them promising for a wide range of practical applications.

### Methods

### Materials and Characterizations

All reagents were purchased from suppliers and used without further purification. ¹H- and ¹³C-NMR spectra were recorded using a Bruker ARX 400 spectrometer where the samples were dissolved in deuterated chloroform at 298 K. Polymer molecular weights were estimated by size exclusion chromatography (SEC) using Agilent 1200 Size Exclusion Chromatography with RI+UV-Vis Detection. For the molecular weight and dispersity characterizations, tetrahydrofuran at 40° C. was used as the mobile phase with molecular weight values based on polystyrene standards. Differential scanning calorimetry (DSC) measurements were performed using a TA Q1000 calorimeter at a heating rate of 10° C. min⁻¹ with nitrogen as the purge gas. Heat-pressing was conducted using Dulytek Elite DE10K Electric Hybrid Rosin Press. The extrusion of the polymers was conducted with a Xplore MC 5 Micro Compounder with the 5 ml batch volume and 1.75 mm extruder. Tensile stress-strain tests were conducted using ADMET MTESTQuattro Universal Testing Machine.

### Synthesis and Processing Procedures

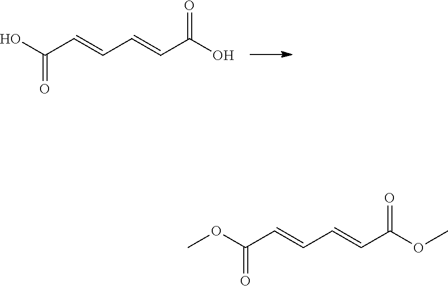

### Synthesis of Trans, Trans-Dimethyl Muconate (ME-Me)

In a 500 mL round-bottom flask with a condenser, Trans, trans-muconic acid (20 g, 0.140 mol) was suspended in 200 ml of methanol. Concentrated H₂SO₄ (2 mL) was added as a catalyst and the mixture was refluxed for 24 hours. The suspension was allowed to cool to room temperature. The crude product was filtered and washed with methanol, following by recrystallization in chloroform and methanol. Dimethyl muconate ester (22 g, 0.129 mol) was obtained as off-white plate crystals after crystallization while cooling in an ice batch with 92% yield. (EE)-ME-Me: ¹H NMR (400 MHZ, CDCl₃, ppm) δ: 7.37-7.27 (m, CH═CHCO₂R, 2H), 6.25-6.15 (m, CH═CHCO₂R, 2H), 3.79-3.15 (s, OCH₃, 3H).

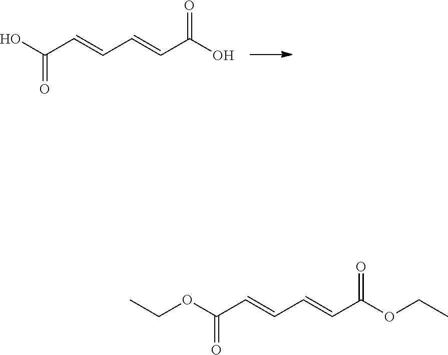

### Synthesis of Trans, Trans-Diethyl Muconate (ME-Et):

In a 500 mL round-bottom flask with a condenser, trans, trans-muconic acid (20 g, 0.140 mol) was suspended in 200 mL of ethanol. Concentrated H₂SO₄ (2 mL) was added as a catalyst and the mixture was refluxed for 24 hours. Then, the solution was allowed to cool to room temperature. The crude product was filtered and washed with water, following by recrystallization in methanol and water. Dimethyl muconate ester (25 g, 0.126 mol) was obtained as off-white plate crystals after crystallization while cooling in an ice batch with 90% yield (EE)-ME-Et: ¹H NMR (400 MHZ, CDCl₃, ppm) δ: 7.37-7.26 (m, CH═CHCO₂R, 2H), 6.23-6.13 (m, CH═CHCO₂R, 2H), 4.26-4.19 (q, OCH₂, 4H), 1.34-1.24 (t, CH₃, J=7.1 Hz, 6H).

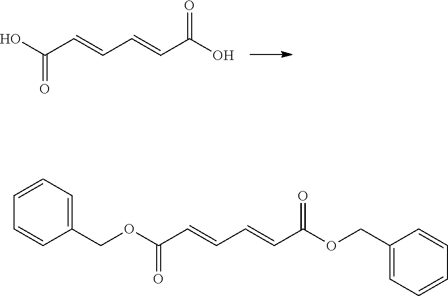 [SMILES: O=C(O)/C=C/C=C/C(=O)O ; O=C(/C=C/C=C/C(=O)OCc1ccccc1)OCc1ccccc1]

### Synthesis of Trans, Trans-Dibenzyl Muconate (ME-B):

In a 500 mL round-bottom flask with a condenser and a dean stark apparatus, trans, trans-muconic acid (20 g, 0.140 mol) was suspended in 200 ml of benzyl alcohol. Concentrated H₂SO₄ (2 mL) was diluted in 2 ml of water and then added as a catalyst and the mixture was refluxed for 24 hours. Then, the solution was allowed to cool to room temperature. The crude product was filtered and washed with methanol, following by recrystallization in chloroform and methanol. 2Dimethyl muconate ester (22 g, 0.069 mol) was obtained as off-white needle crystals after crystallization while cooling in an ice batch with 49% yield. (EE)-ME-B: ¹H NMR (400 MHZ, CDCl₃, ppm) δ: 7.43-7.29 (m, CH═CHCO₂R and C₆H₅, 12H), 6.29-6.19 (m, CH═CHCO₂R, 2H), 5.25-5.19 (s, OCH₂, 4H).

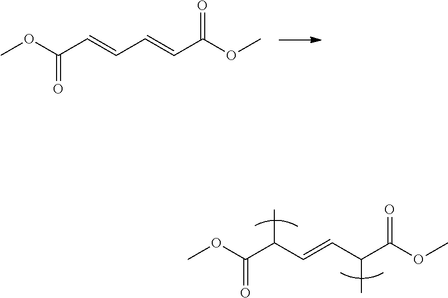

### Polymerization of ME-Me:

In a 50 mL Schlenk tube with a stir bar, dimethyl muconate (5 g, 0.03 mol) was suspended in 14.6 mL anisole. The reaction mixture was flushed with nitrogen for 15 mins and then heated to 120° C. to dissolve all monomer. A stock solution of di-tert-butyl peroxide was prepared by dissolving 27 μL of di-tert-butyl peroxide in 5 mL anisole. Then, 0.1 mL of the stock solution was added to the flask. The reaction mixture was heated at 120° C. for 48 h. The polymer was precipitated 3 times in hexane to remove anisole and the unreacted monomer. Polymer yield is determined by gravimetric method (1.55 g, 31%)

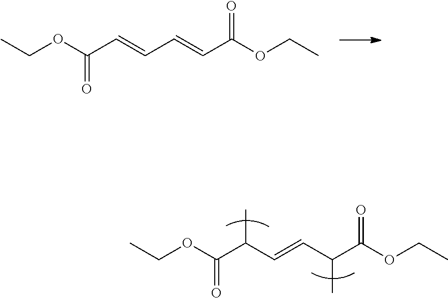

### Polymerization of ME-Et:

In a 25 mL Schlenk tube with a stir bar, diethyl muconate (5 g, 0.025 mol) was dissolved in 12.5 mL anisole. The reaction mixture was flushed with nitrogen for 15 mins and then heated to 120° C. A stock solution of di-tert-butyl peroxide was prepared by dissolving 23 μL of di-tert-butyl peroxide in 5 mL anisole. Then, 0.1 mL of the stock solution was added to the flask. The reaction mixture was heated at 120° C. for 48 h. The polymer was precipitated 3 times in hexane to remove anisole and the unreacted monomer. Polymer yield is determined by gravimetric method (3.5 g, 70%)

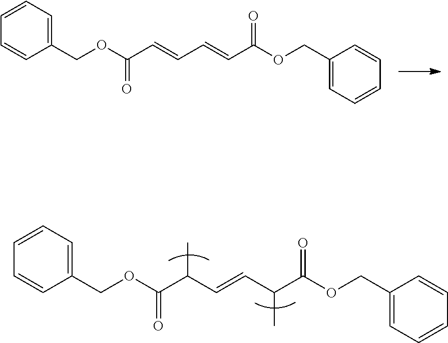

### Polymerization of ME-B:

In a 25 mL Schlenk tube with a stir bar, dibenzyl muconate (5 g, 0.015 mol) was suspended in 7.6 mL anisole. The reaction mixture was flushed with nitrogen for 15 mins and then heated to 120° C. to dissolve all monomer. A stock solution of the di-tert-butyl peroxide was prepared by dissolving 28 UL of di-tert-butyl peroxide in 10 mL anisole. Then, 0.1 mL of the stock solution was added to the flask. The reaction mixture was heated at 120° C. for 48 h. The polymer was precipitated 3 times in methanol to remove anisole and the unreacted monomer. Polymer yield is determined by gravimetric method (3.3 g, 66%)

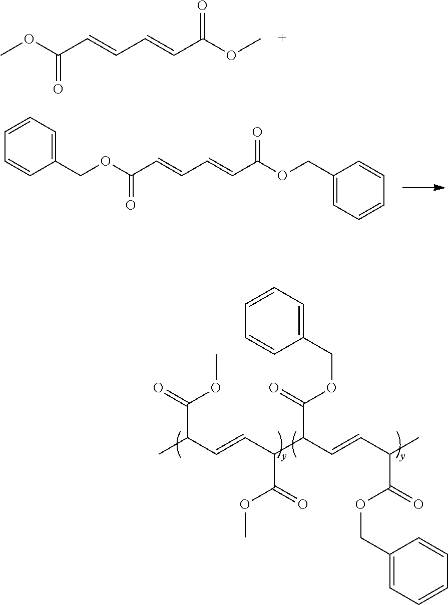

### Copolymerization of ME-B and ME-Me (Polyme-Me3B7):

In a 150 mL Schlenk tube with a stir bar, dimethyl muconate (5 g, 0.029 mol) and dibenzyl muconate (22.1 g, 0.069 mol) was suspended in 39.1 mL anisole. The reaction mixture was flushed with nitrogen for 15 mins and then heated to 120° C. to dissolve all monomer. A stock solution of the di-tert-butyl peroxide was prepared by dissolving 36 μL of di-tert-butyl peroxide in 1 mL anisole. Then, 0.1 mL of the stock solution was added to the flask. The reaction mixture was heated at 120° C. for 48 h. The polymer was precipitated 3 times in methanol to remove anisole and the unreacted monomer. Polymer yield is determined by gravimetric method (7.5 g, 34%)

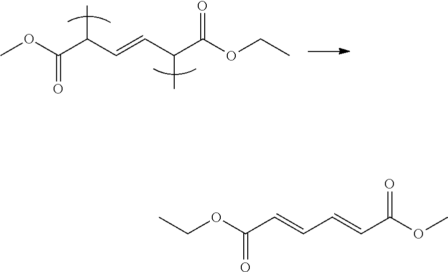

Depolymerization of polyME-Me:

PolyME-Me (50 mg) was mixed with diphenyl ether (10 mL) in a 50 mL round-bottom flask with a condenser under argon atmosphere. The reaction mixture was heated at reflux (˜258° C.) for 45 min. The reaction setup was cooled down quickly by water (to prevent from possible thermal polymerization-depolymerization equilibrium). The solvent diphenyl ether was removed by a short silica column (CH₂Cl₂:Ethyl Acetate=15:1) to obtain crude ME-Me with EE (major), EZ and ZZ isomers (42% crude yield).

The depolymerization of the melt-extruded polyME-Me filament were conducted with the same method as polyME-OMeB crystals with recovery yields of 52%.

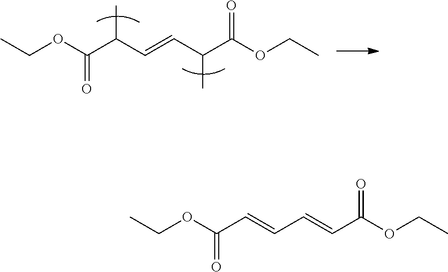

### Depolymerization of PolyME-Et:

PolyME-Et (50 mg) were mixed with diphenyl ether (10 mL) in a 50 mL round-bottom flask with a condenser under argon atmosphere. The reaction mixture was heated at reflux (˜258° C.) for 45 min. The reaction setup was cooled down quickly by water (to prevent from possible thermal polymerization-depolymerization equilibrium). The solvent diphenyl ether was removed by a short silica column (CH₂Cl₂) to obtain crude ME-Et with EE (major), EZ and ZZ isomers (71% crude yield).

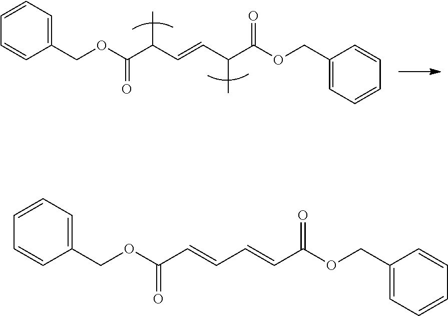

### Depolymerization of PolyME-B:

PolyME-B (50 mg) were mixed with diphenyl ether (10 mL) in a 50 mL round-bottom flask with a condenser under argon atmosphere. The reaction mixture was heated at reflux (˜258° C.) for 45 min. The reaction setup was cooled down quickly by water (to prevent from possible thermal polymerization-depolymerization equilibrium). The solvent diphenyl ether was removed by a short silica column (CH₂Cl₂:Ethyl Acetate=15:1) to obtain crude ME-B with EE (major), EZ and ZZ isomers (67% crude yield)

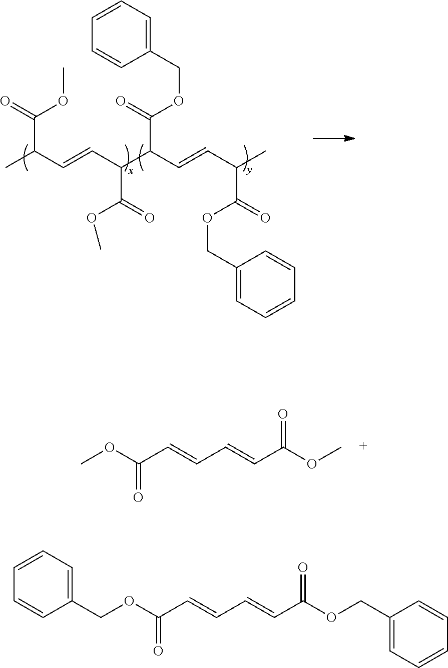

### Depolymerization of PolyME-Me3B7:

PolyME-Me3B7 (50 mg) were mixed with diphenyl ether (10 mL) in a 50 mL round-bottom flask with a condenser under argon atmosphere. The reaction mixture was heated at reflux (˜258° C.) for 45 min. The reaction setup was cooled down quickly by water (to prevent from possible thermal polymerization-depolymerization equilibrium). The solvent diphenyl ether was removed by a short silica column (CH₂Cl₂:Ethyl Acetate=15:1) to obtain crude ME-B/ME-Me with EE (major), EZ and ZZ isomers (61% crude yield)

### Extrusion and 3D Printing of PolyME-Me:

PolyME-Me solid (5.0 g) were fed into a microcompounder which was set at 120° C. The polymer was molten and stable in the microcompounder. The off-white polymer filament with a diameter of roughly 1.6 mm was extruded and collected. The extruded polymer filament was then fed into the 3D printer to print the “P” shape object.

### Example 2

Computational analysis of the polyME structure indicates a relatively low bond dissociation energy for the C—C bond between monomers, even in absence of crystal lattices. This potentially makes the solution phase synthesis of polyME an appealing candidate for circular utilization. The previous study indicates that the cis-cis muconate ethyl ester monomer (Z, Z-ME-Et) can undergo topochemical polymerization with high recyclability. In this work, trans-trans muconate ethyl ester (E, E-ME-Et) is used as a representative monomer for experimental verification of the weakened C—C bond in the polymer prepared by traditional polymerization method. Depolymerization experiments were executed utilizing a solvent-based depolymerization methodology. PolyME-Et underwent conversion to monomers under a temperature of 258° C. within 30 minutes in a diphenyl ether (DPE) solution under reflux. The monomer product was found to be a mixture of cis-cis, cis-trans, and trans-trans configuration isomers present in a ratio of 1:20:80. These isomers can either be directly repolymerized, or converted to a thermodynamically stable E, E-ME isomer via a simple iodine treatment.

When compared to previously reported depolymerization from polymer crystals, free-radical polymerized polyME-Et results in slightly lower yields. From a quantitative ¹³C-NMR study, it is found that this type of polyME contains three repeating unit structures, with partially cis-1,4 addition, trans-1,4 addition, and 1,2 addition, which is a commonly observed phenomenon for butadiene backbone polymers (FIGS. 1B-1C). Due to the double bond conjugation and radical stabilization by the side chain, it is found that the thermodynamically favored trans-1,4 addition contributes 88% of repeating units in polyME, while the cis-1,4 addition contributes 10% and the 1,2 addition contributes a minor 2%. On the other hand, the stereo-irregularity of side chains causes polyME to contain roughly a 50:50 ratio of meso to racemo repeat units. Compared with polyME crystal, which is found to have fully racemo trans-1,4 addition, these polymer structural defects generated from radical polymerizations may contribute to the lowered yield of depolymerization. Calculations show an abnormally high bond dissociation energy (BDE) of the C—C bond between one specific conformation, the trans-1,4+1,2 addition, potentially due to the lack of radical stabilization effect by one double bond (FIG. 1D). By modifying the 1,2-addition ratio of polyME via a preheating treatment, a monotonic decrease in the recycling yield is observed, caused by the significant increase of unreacted oligomers under the same reaction conditions. This indicates that the 1,2-addition can be considered a defect that reduces recycling yield and should be avoided during polymerization. This finding prompts us to dive deeper into the dynamics of polymerization and depolymerization for other polyME derivatives with varied side chain chemistries and functionalities.

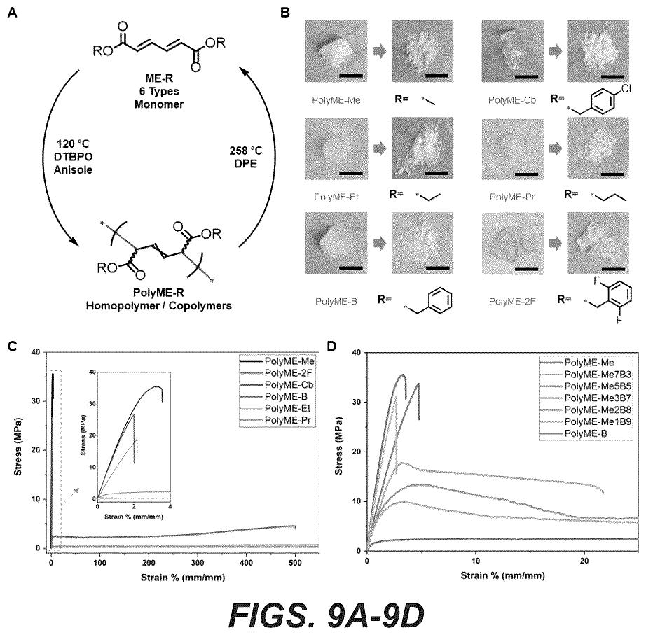

Initial research on polyME homopolymers was carried out by Matsumoto et al. and Quinten et al., but a comprehensive study and copolymerization has not been conducted. In this instance, six polyME derivatives featuring methyl, ethyl, propyl, benzyl, chlorobenzyl, and difluorobenzyl (-Me, -Et, —Pr, —B, —Cb, -2F) were prepared via a modified free-radical polymerization method for experimental validation of polymerization and depolymerization (FIG. 9A). Importantly, all these E, E-ME monomers cannot undergo topochemical polymerization in their single crystal forms and the corresponding polymer crystals have not been accessible so far. Consequently, free-radical polymerization clearly provides a new molecular engineering route with side chain tuning for polyME systems. With a small amount of a tertbutyl peroxide initiator and 48 hours of heating at 120° C., these polymers were obtained with yields of up to 70% (FIGS. 9A-9B). The number average molecular weight of these polymers ranged between 110 kDa to 306 kDa, with the polydispersity index (PDI) between 1.43-1.91 (Table 3).

TABLE 3
Number average molecular weights, PDI, glass transition
temperatures and the corresponding depolymerization
conditions and yields for polyMEs.

| | Number | | | | |
|---|---|---|---|---|---|
| | average | | Glass | Copolymer- | Monomer |
| | molecular | | Transition | ization | Recover |
| | weight | | Temperature | ratio | yield |
| | (kDa) | PDI | (° C.) | (ME:B) | (%) |
| PolyME-Me | 118 | 1.63 | 60 | — | 59 |
| PolyME-Et | 110 | 1.56 | 7 | — | 78 |
| PolyME-Pr | 209 | 1.64 | −17 | — | 75 |
| PolyME-B | 306 | 1.53 | 24 | — | 75 |
| PolyME-Cb | 188 | 1.91 | 34 | — | 80 |
| PolyME-2F | 210 | 1.72 | 52 | — | 82 |
| PolyME- | 155 | 1.67 | 46 | 70:30 | 65 |
| Me7B3 | | | | | |
| PolyME- | 147 | 1.78 | 37 | 50:50 | 63 |
| Me5B5* | | | | | |
| PolyME- | 187 | 1.65 | 32 | 30:70 | 68 |
| Me3B7 | | | | | |
| PolyME- | 192 | 1.73 | 28 | 20:80 | 72 |
| Me2B8 | | | | | |
| PolyME- | 208 | 1.80 | 26 | 10:90 | 70 |
| Me1B9** | | | | | |

*Structure is the same as PolyME-Me7B3, while the repeating unit ratio between Me and B shift from 7:3 to 5:5.
**Structure is the same as PolyME-Me7B3, while the repeating unit ratio between Me and B shift from 7:3 to 1:9.

The wide scope of the molecular weight of polyMEs is attributed to the difference of the molecular weight between each monomer, given that the number of repeating units in different polyMEs are comparable. The molecular weights of these polyMEs are similar to those of commercial polymers such as polymethyl methacrylate (PMMA) and polystyrene (PS). Furthermore, random copolymers composed of various ME-Me and ME-B ratios (70:30, 50:50, 30:70, 20:80, 10:90) were prepared, as copolymerization is a commonly used method for tuning polymer mechanical properties⁴⁹. The ratio of repeating units in polyME copolymers were characterized via ¹H-NMR, and the copolymers were shown to exhibit a roughly similar repeat unit ratio to the monomer feeding ratio (e.g., a 50:50 ME-Me:ME-B monomer feed ratio leads to a formation of 50:50 -Me to —B repeating unit ratio in copolymer, polyME-Me5B5).

All polymers underwent conversion to monomers under the same reaction conditions as polyME-Et. Off-white monomer powders were recovered with yields exceeding 75% for five of the polyME scenarios, and with a yield of 59% for the polyME-Me. Among all polymers, polyME-Me demonstrated the lowest recovery yield, which is attributed to the unstable nature and easy sublimation of the ME-Me monomer. polyME copolymers comprised of ME-Me and ME-B had a recovery yield around 68% for different copolymer ratio (Table 3). Interestingly, ¹H-NMR analysis suggests that the recovered monomers have the same ratio as the polymer composition, making them a suitable candidate to directly synthesize the exact copolymer without supplement of either one of the monomers. The depolymerization findings validate theoretical studies, affirming that the C—C bonds in the polyME derivatives are intrinsically weak, regardless of their polymerization method, copolymerization connectivity, and crystalline/amorphous state.

Differential scanning calorimetry (DSC) was deployed to determine possible phase transitions of polyMEs during heating-cooling cycles and also the tunability of polyME copolymers. No melting could be found within a range of −40 to 200° C., while glass transition temperatures (T_{g}) of polyMEs were detected between −17 to 60° C. (Table 3). This indicates polyMEs made via free radical polymerization are fully amorphous, in agreement with the stereo and conformational irregularity. Interestingly, polyME-Et, with only one more carbon compared with polyME-Me in the side chains, exhibits a rather low T_{g} of 10° C., considering the most rigid polyME-Me has a highest T_{g} of 60° C. among all the homopolymers. The trend of polyMEs' glass transition temperatures based on different side chains is analogous to that of the poly (methyl acrylate) derivative. For polyME copolymers, the glass transition temperatures increase following the Fox equation as the ratio of the ME-Me repeating unit increases. These results indicate that free-radical polymerization is an effective approach to synthesize high quality polyME derivatives with a large range of applications and a wide processing window.

The foundational mechanical properties of polyMEs were evaluated through tensile stress-strain analyses. Large batches of polyME derivatives were produced via a microcompounder and subsequently molded under vacuum heat-pressing to yield standard dog-bone shaped specimens for testing. These polymer samples are optically clear due to their amorphous constitution. PolyME-Me revealed a substantial Young's modulus, averaging 1.52 Gpa, and a peak strength averaging 38 Mpa, due to its rigid and brittle behavior (FIG. 9C). PolyME-B displayed high elongation, with an average strain at break of 462%, along with an average Young's modulus of 0.49 Gpa. PolyME-Et exhibited an exceptionally high elongation, with an average strain at break of 2,170%, and a comparatively low Young's modulus of 0.009 Gpa, which aligns with its low glass transition temperature (T_{g}) and viscoelastic nature. This outcome suggests its potential use as an elastomer after crosslinking, given that the polymer backbone is comparable with butadiene-based natural or synthetic rubber. The copolymers of ME-Me and ME-B exhibited characteristics deriving from both polyME-Me and polyME-B, as shown in FIG. 9D, which can either maintain a robust Young's modulus of 1.41 Gpa with a higher elongation percentage than polyME-Me (polyME-Me5B5), or have a partially decreased Young's modulus with moderate elongation of 19.4% (polyME-Me3B7). Notably, these findings reveal that the mechanical properties of copolymers do not evolve linearly with the monomer ratios, due to the polyME-Me component largely dominates the mechanical properties. The divergent mechanical behaviors of the various polyMEs can be achieved by mere modification of the side chain chemistry and copolymerization, which illustrates the extensive application potential of these recyclable polymers compared to commercial polymers.

To further demonstrate the potential of polyME in larger scale synthesis, more than 400 grams of monomer (FIG. 10A-10B) is gained in one batch reaction. A hundred-gram scale synthesis of PolyME-Me3B7 was performed to test large scale polymerization and polymer processing. Size exclusion chromatography (SEC) of the synthesized polymer indicated nearly zero decrease in molecular weight (FIG. 10C). Polymer filaments were prepared by extruding at a moderate temperature of 120° C. with a microcompounder, which yielded a diameter of ˜1.75 mm (FIG. 10D). The filament was subsequently fed into a 3D printer to produce a Purdue “P” logo. To demonstrate that polyMEs can be processed by commercial processing methods, a plastic dinosaur was created with injection molding with polyME-Me3B7 (FIG. 10E).

With the successful laboratory scale-up, further exploration into the potential for large scale production of polyME was considered. Although E,E-muconic acid can be derived from petroleum resources such as arene rings, adipic acid and catechol, these synthetic pathways requires either metal catalyst or thionyl chloride. On the other hand, the bio-derived muconic acid can be obtained from bacteria via evolution and metabolic engineering. The bio-derived muconic acid exclusively exists in the cis-trans (E,Z) configuration, leading to a formation of E, Z-ME. This monomer undergoes the same free radical polymerization process, resulting in E,Z-polyME-Me with similar molecular weight. Since the depolymerization product thermodynamically favors the E,E monomer, the result depolymerization product of E,Z-polyME-Me is identical to the product from E,E-polyME-Me. This result indicates that the bio-derived ME can be integrated into the closed-loop recycling cycle of polyME after a first life without the need of any further treatment.

Free-radical polymerization serves as an effective method for the generation of high-molecular-weight polyME derivatives and copolymers. Similar to the topochemically prepared polymer crystals, these polymers retain the capability for depolymerization under thermolysis approximately at 250° C. within a high boiling point solvent, attributing to their intrinsically weakened C—C bonds. Intriguingly, merely altering the side groups permits the mechanical properties of these polymers to transition from hard and fragile to soft and extendable. This basic design rules also suggest that a large space of tensile-strained polymers potentially exists, which could be explored with ascendant machine-learning based approaches. For example, new reversible backbones can be searched, new functional groups can be added, copolymer and block-copolymer can be synthesized, polymer blends and composites can be manufactured, potentially new polymer physics is associated with these materials, and more industrial relevant processing techniques (e.g., injection-molding, extrusion, and 3D printing) can be developed. Moreover, the fundamental chemistry deployed here is scalable, and the processing of these materials is fully harmonious with extant industry frameworks. TEA and LCA results highlight the potential of production of polyME-Et from bio-based muconic acid and its recyclability. The economic and environmental metrics evaluated for polyME-Et are comparable with conventional PMMA, and combining the depolymerization significantly lowers the MSP, SCE, and GHG emissions. Thus, this work will have a profound impact on the broad fields of organic chemistry, polymer science, environmental science, as well as the commodity chemical and plastics industries.

What is claimed is:

1. A process for obtaining a monomer of formula (M-I) and/or a monomer of formula (M-II) 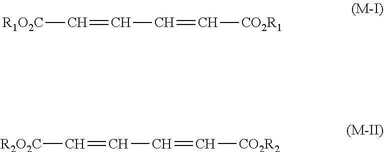
from a non-crystalline random co-polymer of formula (P-I) 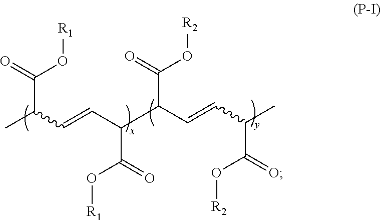
or
a monomer of formula (M-I):
wherein:
x indicates the mole percentage (mol %) of the repeating unit R₁O₂C—CH—CH═CH—CH—CO₂R₁ in P-I;
y indicates the mol % of the repeating unit R₂O₂C—CH—CH═CH—CH—CO₂R₂) in P-I,
where x+y=100 with the proviso that if y=0, only the monomer (M-I) is obtained and if x=0, only the monomer (M-II) is obtained; and
R₁ and R₂ are not identical and are independently selected from the group consisting of alkyl and optionally substituted aryl-alkylene;
the process comprising the steps of heating the polymer of formula (P-I) in a solvent under an inert atmosphere at a temperature of about 240° C. to about a temperature of about 270° C. for a time of about 30 minutes to about 60 minutes, cooling the solution to about room temperature; and isolating the M-I, M-II, or the mixture of M-I and M-II by chromatography.

2. The process of claim 1, wherein R₁ and R₂ are each independently alkyl or R₁ and R₂ are each independently optionally substituted aryl-alkylene.

3. The process of claim 1, wherein R₁ is C₁-C₄ alkyl, C₁-C₈ alkyl, or C₁-C₈ alkyl.

4. The process of claim 1, wherein R₂ is C₁-C₆ alkyl, C₁-C₈ alkyl, or optionally substituted aryl-alkylene, with the proviso that R₁ and R₂ are different.

5. The process of claim 1, wherein R₂ is optionally substituted aryl-alkylene.

6. The process of claim 1, wherein the solvent selected from group consisting of 1,3-dimethyltetrahydropyrimidin-2(1H)-one (DMPU), 2,5,8,11,14-Pentaoxapentadecane (TEGDME), and diphenyl ether.

7. The process of claim 1, wherein the temperature is about 250° C. to about 280° C.

8. The process of claim 1, wherein the time is about 45 minutes.

9. The process of claim 1, wherein y is 0 mol % to 100 mol %.

10. The process of claim 9, wherein y is about 70 mol %.

11. The process of claim 1, wherein the obtained monomer (M-I) and/or (M-II) is the E,E isomer, substantially free of the E,Z and Z,Z isomers.

12. A non-crystalline random co-polymer comprising a first repeating unit of the chemical formula (R₁O₂C—CH—CH═CH—CH—CO₂R₁)ₓ and second repeating unit of the chemical formula (R₂O₂C—CH—CH═CH—CH—CO₂R₂)ᵥ
wherein:
x indicates the non-zero mole percentage (mol %) of the first repeating unit;
y indicates the non-zero mol % of the second repeating unit; and
R₁ and R₂ are not identical and are independently selected from the group consisting of alkyl and optionally substituted aryl-alkylene.

13. The non-crystalline random co-polymer of claim 12, wherein R₁ and R₂ are independently alkyl or R₁ and R₂ are independently optionally substituted aryl-alkylene.

14. The non-crystalline random co-polymer of claim 12, wherein R₁ is C₁-C₄ alkyl, C₁-C₆ alkyl, or C₁-C₈ alkyl.

15. The non-crystalline random co-polymer of claim 12, wherein R₂ is C₁-C₆ alkyl, C₁-C₈ alkyl, or optionally substituted aryl-alkylene.

16. The non-crystalline random co-polymer of claim 12, wherein y is 5 mol % to 95 mol %.

17. The non-crystalline random co-polymer of claim 12, wherein y is about 50 mol % to about 90 mol %.

18. The non-crystalline random co-polymer of claim 12, wherein the non-crystalline random co-polymer is a thermoplastic.

19. The non-crystalline random co-polymer of claim 12, wherein the non-crystalline random co-polymer has a Young's Modulus of about 0.5 to about 1.6.

20. The non-crystalline random co-polymer of claim 12, wherein the non-crystalline random co-polymer has a weight average molecular weight (Mw) from about 100 kDa to about 600 kDa; an ultimate strength of from about 1 MPa to about 45 MPa; and/or a strain at break of from about 2% to about 2500%.
